(19) United States
(12) Patent Application Publication
(10) Pub. No.: US 2026/0053854 A1
(43) Pub. Date: Feb. 26, 2026

(54) ANTI-PSMA SINGLE-CHAIN ANTIBODY, CHIMERIC ANTIGEN RECEPTOR ASSOCIATED THEREWITH AND USE THEREOF

(71) Applicant: BEIJING MEIKANG GENO-IMMUNE BIOTECHNOLOGY CO., LTD., Beijing (CN)

(72) Inventor: Lung-ji Chang, Beijing (CN)

(73) Assignee: BEIJING MEIKANG GENO-IMMUNE BIOTECHNOLOGY CO., LTD., Beijing (CN)

(21) Appl. No.: 19/102,569

(22) PCT Filed: Aug. 15, 2023
(86) PCT No.: PCT/CN2023/113083

§ 371 Date: Feb. 10, 2025

(30) Foreign Application Priority Data
Aug. 17, 2022 (CN) 202210987514.5

(51) Int. Cl. A61K 35/17; A61K 40/11; A61K 40/31; A61K 40/42; A61P 35/00; C07K 14/705; C07K 14/715; C07K 14/725; C07K 16/30; C12N 5/0783; C12N 9/64; C12N 15/86

(52) U.S. Cl. CPC A61K 35/17; A61K 40/11; A61K 40/31; A61K 40/4276; A61P 35/00; C07K 14/7051; C07K 14/70517; C07K 14/70521; C07K 14/70578; C07K 14/7155; C07K 16/3069; C12N 5/0636; C12N 9/6472; C12N 15/86; A61K 2239/13; A61K 2239/21; A61K 2239/25; C07K 2317/622; C07K 2319/02; C12N 2740/15043; C12N 2800/40; C12Y 304/22062

(57) ABSTRACT

Provided are an anti-PSMA single-chain antibody, a chimeric antigen receptor associated therewith and use thereof. An amino acid sequence of a heavy chain of the anti-PSMA single-chain antibody includes a sequence shown in SEQ ID NO: 1, and an amino acid sequence of a light chain of the anti-PSMA single-chain antibody includes a sequence shown in SEQ ID NO: 2. The anti-PSMA single-chain antibody is a humanized scFv antibody of PSMA, is more functional in the human body, has better compatibility, and is less prone to be rejected by the immune system. The chimeric antigen receptor has better response effects after specifically bonding to PSMA so that CAR-T cells generate a stronger immune response to tumors, and the chimeric antigen receptor also has better long-term effectiveness than other PSMA chimeric antigen receptors.

### TECHNICAL FIELD

The present application belongs to the field of tumor immunotherapy and relates to an anti-prostate-specific membrane antigen (PSMA) single-chain antibody, a chimeric antigen receptor associated therewith and use thereof.

### BACKGROUND

With the development of tumor immunology theories and clinical techniques, chimeric antigen receptor T-cell (CAR-T) immunotherapy has become one of the most promising tumor immunotherapies. Generally, a chimeric antigen receptor (CAR) consists of a tumor-associated antigen binding domain, an extracellular hinge region, a transmembrane domain, and an intracellular T cell signaling domain. Generally, the CAR contains a single-chain variable fragment (scFv) region of an antibody or a binding domain having specificity to a tumor-associated antigen (TAA), which is coupled to a cytoplasmic domain of a T cell signaling molecule via hinges and transmembrane domains. The most common lymphocyte activation moiety includes a T cell costimulatory domain in tandem with a moiety (for example, CD3ζ) triggering the function of a T-cell effector.

CAR-mediated adoptive immunotherapy allows CAR-transplanted T cells to directly recognize TAAs on target tumor cells in a non-human leukocyte antigen (non-HLA)-restricted manner. At present, the CAR-T treatment has achieved a certain effect on hematological tumors, and the second-generation CD19 CAR-T cell has demonstrated anti-tumor efficacy in acute B lymphoblastic leukemia, chronic lymphoblastic leukemia, and lymphoma, with an overall response rate of about 50% to 90% depending on the tumors.

The prostate-specific membrane antigen (PSMA) is widely expressed in tumors such as prostate cancers, renal cancers, bladder cancers, glioblastomas, brain tumors, multiple myelomas and B-cell lymphomas and microvessels in the tumor microenvironment, is expressed in low and restricted amounts in normal tissues, and thus is an ideal tumor-associated antigen for immunotherapy. At present, such a cancer antigen is mostly used in the treatment of prostate cancer which is an epithelial malignancy occurring in the prostate and is the common malignancy in the male genitourinary system. However, in addition to prostate tumors, PSMA is also highly expressed in microvessels in the stromal tissues of most tumors.

At present, in the immunotherapy for glioblastomas and brain tumors, the antibody therapy against PSMA has been developed maturely and has achieved preliminary success in the clinic. Antibodies are present in the peripheral blood after administration, hardly accurately enter the tumor tissue or the sites where small numbers of tumors remain, and cannot exist in vivo for a long period of time. Therefore, a chimeric antigen receptor PSMA CAR-T cell is prepared. In addition to having the advantages of antibody therapy, due to the nature of the T cells themselves, the PSMA CAR-T cell can accurately enter the tumor tissue and exist in vivo with a long-term memory, providing a more effective treatment option for patients with recurrent and refractory cancers. Current clinical reports on the treatment of glioblastomas with PSMA CAR-T cells have preliminarily demonstrated the efficacy of CAR-T cells, but long-term observational data are still lacking.

At present, the CAR-T technology is not very effective in treating solid tumors. The scFv region of various CAR-T cells is derived from mouse-derived antibodies, and such a mouse-derived scFv is prone to be rejected by the human immune system so that the CAR-T cell cannot exist in vivo for a long period of time, thereby limiting the therapeutic effect. It has been reported that this is also one of the reasons why many patients with acute lymphoblastic leukemia relapse after being in complete remission with CD19 CAR-T cells, and it also makes retreatment difficult.

Therefore, how to provide a humanized PSMA CAR-T cell with a good therapeutic effect and a long duration has become one of the urgent problems in the field of tumor immunotherapy.

### SUMMARY

To achieve the above objectives, the present application provides an anti-PSMA single-chain antibody (anti-PSMA scFv), a chimeric antigen receptor associated therewith and use thereof. The anti-PSMA single-chain antibody has high specificity and affinity and is capable of efficiently binding to PSMA. Through a PSMA chimeric antigen receptor of the anti-PSMA single-chain antibody and corresponding CAR-T cells, PSMA-positive solid tumors can be eliminated, and the tumor microenvironment can be effectively targeted to remove minimal residues without serious adverse effects. Therefore, the safety, efficacy, memory, and long-term maintainability of the CAR-T cell can be improved.

To achieve the above objectives, the present application adopts the solutions below.

In a first aspect, in the present application, the anti-PSMA single-chain antibody is a humanized scFv antibody of PSMA. The scFv antibody has been specifically modified against the tumor surface antigen PSMA with humanized codons and humanized antibody frameworks, and the modified scFv antibody is more functional in the human body, has a better compatibility, and is less prone to be rejected by the immune system.

In the present application, an amino acid sequence of a heavy chain (VH) of the anti-PSMA single-chain antibody includes a sequence having 80% or more identity to SEQ ID NO: 1, and an amino acid sequence of a light chain (VL) of the anti-PSMA single-chain antibody includes a sequence having 80% or more identity to SEQ ID NO: 2.

SEQ ID NO: 1:
EVQLVQSGAEVKKPGASVKISCKISGYTFTEYTIHWVKQASGKGLEWIGN
INPNNGGTTYNQKFEDRATLTVDKSTSTAYMELSSLRSEDTAVYYCAAGW
NFDYWGQGTTVTVSS.
SEQ ID NO: 2:
DIVMTQSPSSLSASVGDRVTIICKASQDVGTAVDWYQQKPGKAPKLLIYW
ASTRHTGVPDRFTGSGSGTDFTLTISSLQPEDFADYFCQQYNSYPLTFGG
GTKLEIK.

The anti-PSMA scFv VH and the anti-PSMA scFv VL may be connected via a linker, for example, GSTSGSGKPGSSEGSTKG (SEQ ID NO: 12).

In one specific embodiment, the sequence of the
anti-PSMA scFv is SEQ ID NO: 9:
EVQLVQSGAEVKKPGASVKISCKISGYTFTEYTIHWVKQASGKGLEWIGN
INPNNGGTTYNQKFEDRATLTVDKSTSTAYMELSSLRSEDTAVYYCAAGW
NFDYWGQGTTVTVSSGSTSGSGKPGSSEGSTKGDIVMTQSPSSLSASVGD
RVTIICKASQDVGTAVDWYQQKPGKAPKLLIYWASTRHTGVPDRFTGSGS
GTDFTLTISSLQPEDFADYFCQQYNSYPLTFGGGTKLEIK.

In a second aspect, the present application provides a nucleic acid molecule. The nucleic acid molecule includes a nucleic acid sequence encoding the anti-PSMA single-chain antibody described in the first aspect.

Preferably, a nucleotide sequence of the nucleic acid molecule has 80% or more identity to SEQ ID NO: 3.

SEQ ID NO: 3:
gaggtccaactggtgcagtctggtgctgaagtgaagaagcctggtgccag
cgtgaagattagctgcaagattagcggctacaccttcaccgagtatacca
ttcactgggtcaaacaagcctctggaaaagggctggaatggataggcaat
atcaaccccaacaatggcggaacaacctacaaccaaaagtttgaggatcg
tgccactctgacggttgacaagtccacgagcacagcctacatggagctgt
caagtctgaggtccgaggatactgcggtctactattgtgctgctgggtgg
gggtccagtgaagggagcacaaaaggggacatcgtgatgacccagtctcc
aagcagccttagcgctagtgtaggggatcgagtgaccatcatctgcaaag
catctcaggacgtaggcactgcagtggattggtatcagcagaaaccagga
aaagcgccgaaactgttgatctactgggcaagtacacgccacactggagt
cccagatcggtttaccgggtccggctcaggcactgacttcactctgacca
tttcctctcttcagcccgaagattttgccgactacttctgtcagcagtac
aatagctatcccctgacatttggcggtggaacaaagctcgagataaag.

In a third aspect, the present application provides a PSMA chimeric antigen receptor. The PSMA chimeric antigen receptor includes an antigen binding domain, a transmembrane domain, a costimulatory signaling region, and a CD3ζ signaling domain. The antigen binding domain includes the anti-PSMA single-chain antibody described in the first aspect.

The PSMA chimeric antigen receptor of the present application is obtained by performing specific genetic modification on the costimulatory signaling domain of a humanized chimeric antigen receptor targeting the tumor surface antigen PSMA, and the structure of the PSMA chimeric antigen receptor is shown in FIG. 1. The modified chimeric antigen receptor has better response effects after specifically bonding to PSMA so that CAR-T cells generate a stronger immune response to tumors, and the modified chimeric antigen receptor also has better long-term effectiveness than other PSMA chimeric antigen receptors.

Preferably, the transmembrane domain includes a CD28 transmembrane domain and/or a CD8α transmembrane domain.

Preferably, the costimulatory signaling domain includes a CD28 signaling domain and a CD27 signaling domain or includes a CD28 signaling domain and an IL-15Ra signaling domain.

In one specific embodiment, the PSMA CAR includes a CD28 transmembrane domain, a CD28 signaling domain, and a CD27 signaling domain. In this embodiment, the PSMA CAR preferably includes an amino acid sequence having 90% or more identity to SEQ ID NO: 4.

In one specific embodiment, the PSMA CAR includes a CD28 transmembrane domain, a CD28 signaling domain, and an IL-15Ra signaling domain. In this embodiment, the PSMA CAR preferably includes an amino acid sequence having 90% or more identity to SEQ ID NO: 5.

Preferably, the PSMA chimeric antigen receptor further includes a suicide-inducing fusion domain.

Preferably, the suicide-inducing fusion domain includes a caspase 9 domain fused with an FK506 binding protein (FKBP) (such a fused caspase 9 is abbreviated to FKBP.Casp9).

Preferably, an amino acid sequence of the FKBP.Casp9 domain has 90% or more identity to SEQ ID NO: 6.

Preferably, the PSMA chimeric antigen receptor further includes a signal peptide and/or a 2A sequence.

Preferably, the signal peptide includes a Secretory signal peptide.

Preferably, the Secretory signal peptide is a signal peptide of a CD8α gene, and an amino acid sequence of the Secretory signal peptide is: MALPVTALLLPLALLLHAARP (SEQ ID NO: 10). Alternatively, the Secretory signal peptide may be a signal peptide of a GM-CSFR gene, and an amino acid sequence of the Secretory signal peptide is: MLLLVTSLLLCELPHPAFLLIP (SEQ ID NO: 11). Those skilled in the art can select the Secretory signal peptide according to the actual situation, and the Secretory signal peptide is not particularly limited herein. The presence of the Secretory signal peptide has no effect on the performance of the chimeric antigen receptor of the present application.

SEQ ID NO: 4:
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRSASGGGGSGGGGSQRRKYRSNKGESPVEPAEPCHYSCPREEEGS
TIPIQEDYRKPEPACSP (CD28 hinge domain + CD28
transmembrane domain + CD28 signaling domain +
linker + CD27 signaling domain).
SEQ ID NO: 5:
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRSASGGGGSGGGGSKSRQTPPLASVEMEAMEALPVTWGTSSRDED
LENCSHHL (CD28 hinge domain + CD28 transmembrane
domain + CD28 signaling domain + linker + IL-15Ra
signaling domain).
SEQ ID NO: 6:
MGVQVETISPGDGRTFPKRGQTCVVHYTGMLEDGKKVDSSRDRNKPFKFM
LGKQEVIRGWEEGVAQMSVGQRAKLTISPDYAYGATGHPGIIPPHATLVF
DVELLKLEGGGGSGGGGSGAMVGALESLRGNADLAYILSMEPCGHCLIIN
NVNFCRESGLRTRTGSNIDCEKLRRRFSSLHFMVEVKGDLTAKKMVLALL
ELARQDHGALDCCVVVILSHGCQASHLQFPGAVYGTDGCPVSVEKIVNIF
NGTSCPSLGGKPKLFFIQACGGEQKDHGFEVASTSPEDESPGSNPEPDAT
PFQEGLRTFDQLDAISSLPTPSDIFVSYSTFPGFVSWRDPKSGSWYVETL
DDIFEQWAHSEDLQSLLLRVANAVSVKGIYKQMPGCFNFLRKKLFFKTSA
S (FKBP + linker + Casp9).

According to the present application, the signal peptide in the PSMA chimeric antigen receptor is a signal peptide capable of directing the transmembrane transfer of the chimeric antigen receptor, and those skilled in the art can select a secretory protein gene signal peptide conventional in the art as required.

According to the present application, the suicide-inducing fusion domain is connected in tandem with the CD3ζ signaling domain via a 2A sequence. The 2A sequence can cleave the protein expressed by the suicide-inducing fusion domain and the protein of the PSMA chimeric antigen receptor, enabling the chimeric antigen receptor to function. With an activator injected, the suicide-inducing fusion domain is activated, causing the chimeric antigen receptor to become inactive.

In the present application, the chimeric antigen receptor further includes a linker, and an amino acid sequence of the linker is GSTSGSGKPGSSEGSTKG (SEQ ID NO: 12) or is a repetitive combination of a plurality of GGGGS (SEQ ID NO: 13), which may be, for example, GGGGSGGGGS (SEQ ID NO: 14) or GGGGSGGGGSGGGGS (SEQ ID NO: 15). Those skilled in the art can select a linker according to the actual situation, and the linker is not particularly limited herein. The presence of the linker has no effect on the performance of the chimeric antigen receptor of the present disclosure.

Preferably, the PSMA chimeric antigen receptor includes a Secretory signal peptide, an antigen binding domain, a transmembrane domain, a costimulatory signaling domain, a CD3ζ signaling domain, a 2A sequence, and a suicide-inducing fusion domain.

As a preferred solution, the PSMA chimeric antigen receptor is composed of a Secretory signal peptide (Secretory signal), an anti-PSMA single-chain antibody (PSM scFv), CD8α and/or CD28 transmembrane domains, CD28 and CD27 signaling domains, a CD3ζ signaling domain, a 2A sequence, and a caspase 9 domain (FKBP.Casp9) connected in tandem, and the specific arrangement is as follows: Secretory signal-PSM scFv-CD28-CD27-CD3ζ-2A-FKBP.Casp9, where CD28 represents a CD28 extracellular signal structure and a CD28 transmembrane domain and its intracellular signaling domain. Alternatively, the PSMA chimeric antigen receptor is composed of a Secretory signal peptide, an anti-PSMA single-chain antibody (PSM scFv), CD8α and/or CD28 transmembrane domains, CD28 and IL-15Ra signaling domains, a CD3ζ signaling domain, a 2A sequence, and a caspase 9 domain (FKBP.Casp9) connected in tandem, and the specific arrangement is as follows: Secretory signal-PSMA scFv-CD28-IL-15Ra-CD3ζ-2A-FKBP.Casp9, where CD28 represents a CD28 transmembrane domain and its intracellular signaling domain.

Preferably, an amino acid sequence of the CD34 signaling domain includes a sequence shown in SEQ ID NO: 7.

Preferably, an amino acid sequence of the 2A sequence includes a sequence shown in SEQ ID NO: 8.

SEQ ID NO: 7:
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.
SEQ ID NO: 8:
TSGSGATNFSLLKQAGDVEENPGP.

In the present application, the chimeric antigen receptor further includes a promoter, and the promoter is EF1α or any one of highly expressed promoters.

In a fourth aspect, the present application provides a nucleic acid molecule. The nucleic acid molecule encodes the PSMA chimeric antigen receptor described in the third aspect.

In a fifth aspect, the present application provides a viral vector. The viral vector includes a nucleic acid molecule encoding the PSMA chimeric antigen receptor described in the third aspect.

The viral vector includes at least one copy of the nucleic acid molecule described in the fourth aspect.

Preferably, the viral vector includes a lentiviral vector or a retroviral vector and preferably, is a lentiviral vector.

In the present application, the viral vector can effectively modify immune cells to prepare targeted cells.

In a sixth aspect, the present application provides a recombinant virus. The recombinant virus is obtained by co-transduction of the viral vector described in the fifth aspect and packaging helper plasmids into a mammalian cell.

Preferably, the packaging helper plasmids include pNHP and pHEF-VSVG.

Preferably, the mammalian cell includes any one of a 293 cell, a 293 T cell or a TE671 cell.

In a seventh aspect, the present application provides a chimeric antigen receptor cell. The chimeric antigen receptor cell expresses the PSMA chimeric antigen receptor described in the third aspect.

In the present application, the mechanism of action of the chimeric antigen receptor cell is shown in FIG. 1, and the chimeric antigen receptor cell has a great targeted killing effect, releases low doses of immune factors and has a response property of low toxicity and high immune killing.

Preferably, the chimeric antigen receptor cell is prepared by transduction of a nucleic acid molecule encoding the PSMA chimeric antigen receptor described in the third aspect into an immune cell.

Preferably, the manner of the transduction includes any one of transduction by a viral vector, transduction by a eukaryotic expression plasmid or transduction by mRNA and preferably, is transduction by a viral vector.

Preferably, the immune cell includes a T cell.

In an eighth aspect, the present application provides a composition. The composition includes any one or a combination of at least two of the anti-PSMA single-chain antibody described in the first aspect, the nucleic acid molecule described in the second aspect, the PSMA chimeric antigen receptor described in the third aspect, the viral vector described in the fifth aspect, the recombinant virus described in the sixth aspect or the chimeric antigen receptor cell described in the seventh aspect.

In a ninth aspect, the present application provides use of any one or a combination of at least two of the anti-PSMA single-chain antibody described in the first aspect, the nucleic acid molecule described in the second aspect, the PSMA chimeric antigen receptor described in the third aspect, the viral vector described in the fifth aspect, the recombinant virus described in the sixth aspect, the chimeric antigen receptor cell described in the seventh aspect or the composition described in the eighth aspect in the preparation of a drug for treating a tumor.

Preferably, the tumor includes a tumor expressing a PSMA-specific antigen.

Preferably, the tumor includes a prostate cancer, a lymphoma, a renal cancer, a bladder cancer, a colon cancer, a neuroblastoma or a brain tumor.

Compared with the existing art, the present application has the beneficial effects described below.

(1) The anti-PSMA single-chain antibody of the present application is a humanized scFv antibody of PSMA. The scFv antibody is specifically modified against the tumor surface antigen PSMA with humanized codons and humanized antibody frameworks, and the modified scFv antibody is more functional in the human body, has a better compatibility, and is less prone to be rejected by the immune system.

(2) The chimeric antigen receptor of the present application is obtained by performing specific genetic modification on the costimulatory signaling domain of a humanized chimeric antigen receptor targeting the tumor surface antigen PSMA. The modified chimeric antigen receptor has better response effects after specifically bonding to PSMA so that CAR-T cells generate a stronger immune response to tumors, and the modified chimeric antigen receptor also has better long-term effectiveness than other PSMA chimeric antigen receptors.

(3) The chimeric antigen receptor T cell of the present application has higher safety and durability than other PSMA chimeric antigen receptor T cells. Even if an overly strong immune response occurs and causes an immune factor storm in patients, due to the apoptosis-inducing mechanism, the chimeric antigen receptor T cell can be withdrawn by a drug inducing CAR-T cell apoptosis. After the chimeric antigen receptor T cell of the present application is infused, the presence of the CAR-T cell can be monitored in vivo for a long period of time, which proves that the chimeric antigen receptor T cell has long-term effectiveness and can achieve long-term remission in patients.

(4) The humanized antibody-associated preparation in the present application can work against all PSMA-positive diseases, has been practically applied in patients with tumors expressing the tumor-specific target PSMA, has fewer clinical side effects and higher safety, and can effectively eliminate the minimal residues that are insensitive to chemotherapy. In addition, the PSMA CAR-T cells are also used in combination with other targeted CAR-T cells in patients with brain tumors, and the presence of the PSMA CAR-T cells is monitored in patients in vivo for a long period of time, which is conducive to maintaining long-term remission.

### BRIEF DESCRIPTION OF DRAWINGS

FIG. 1 is a schematic diagram showing the structure of a chimeric antigen receptor and the mechanism of action of the chimeric antigen receptor T cell;

FIG. 2 is a diagram showing in vitro killing of PSMA CAR-T cells against PSMA-positive tumor cell lines;

FIG. 3A is a schematic diagram showing the process of treating brain glioma with PSMA CAR-T cells;

FIG. 3B is an image showing immunohistochemical staining results of tumor sections of a patient with brain glioma (with a magnification factor of 20×);

FIG. 4 is a curve graph of CAR copy numbers detected in vivo after PSMA CAR-T cells are infused in Example 8; and

FIG. 5 is an MRI graph of changes in brain tumor lesions before and after the infusion of PSMA CAR-T cells.

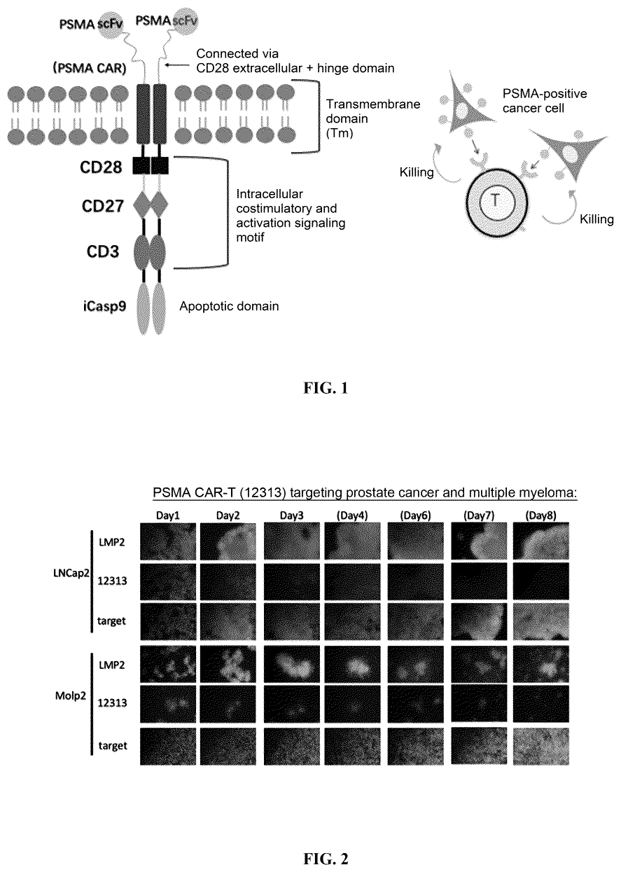

### DETAILED DESCRIPTION

To further elaborate on the technical means adopted and effects achieved in the present application, the present application is further described below in conjunction with examples and drawings. It is to be understood that the specific examples set forth below are intended to explain the present application and not to limit the present application.

Experiments without specific techniques or conditions specified in the examples are conducted according to techniques or conditions described in the literature in the art or according to product specifications. The reagents or instruments used herein without manufacturers specified are conventional products commercially available from proper channels.

### Example 1

This example provided a humanized scFv antibody of PSMA. The humanized scFv antibody of PSMA had the activity of binding to a PSMA antigen.

The nucleic acid sequence of the humanized scFv antibody of PSMA was shown in SEQ ID NO: 3.

### Example 2

This example provided a chimeric antigen receptor, where the chimeric antigen receptor was composed of a Secretory signal peptide (MALPVTALLLPLALLLHAARP (SEQ ID NO: 10)), an anti-PSMA single-chain antibody (SEQ ID NO: 9), a CD28 transmembrane domain, CD28 and CD27 signaling domains (SEQ ID NO: 4), a CD3ζ signaling domain (SEQ ID NO: 7), a 2A sequence (SEQ ID NO: 8), and a caspase 9 domain (SEQ ID NO: 6) connected in tandem, and the specific arrangement was as follows: secretory signal-PSMA scFv-CD28-CD27-CD32-2A-FKBP.Casp9, where the CD28 represented a CD28 transmembrane domain and its intracellular signaling domain. The chimeric antigen receptor was named the chimeric antigen receptor 12313.

### Example 3

This example provided a lentiviral vector. The lentiviral vector encoded the chimeric antigen receptor in Example 2.

The backbone vector of the lentiviral vector was pTYF, and for details, see Chang, L.-J. and Zaiss, A.-K. (2001) Methods for the preparation and use of lentivirus vectors. Methods in Molecular Medicine, Gene Therapy Protocols, 2nd Ed., pp 303-318, Ed. Jeffrey Morgan, Humana Press, Inc.; Cui, Y. and Chang, L.-J. (2003) Detection and selection of lentiviral vector transduced cells. “Methods in Molecular Biology Vol. 229: Lentivirus Gene Engineering Protocols” pp 69-85, Ed. Maurizio Federico, Humana Press, Inc; Oka, M. Chang, L.-J., Costantini, F., and Terada, N. (2005) Lentivirus mediated gene transfer in embryonic stem cells. Series: “Methods in Molecular Biology” Embryonic Stem Cells 2.

### Example 4

This example provided a recombinant lentiviral vector. The recombinant lentiviral vector was obtained by co-transducing a mammalian cells with the lentiviral viral vector described in Example 3, or a control viral protein (EBV LMP 2) antibody CAR lentivirus vector not associated with target cells, and packaging helper plasmids, and the steps are as follows:

(1) 293T cells were cultured for 18 h.

(2) A fresh DMEM (purchased from Thermo Fisher) was added.

(3) The following reagents were added to a sterile centrifuge tube: each well was charged with a DMEM, packaging helper plasmids (pNHP and pHEF-VSV-G), and a pTYF CAR DNA vector (for specific operations, see Chang, L.-J. and Zaiss, A.-K. (2001) Methods for the preparation and use of lentivirus vectors. Methods in Molecular Medicine, Gene Therapy Protocols, 2nd Ed., pp 303-318, Ed. Jeffrey Morgan, Humana Press, Inc.), and then the tube was vortexed and oscillated.

(4) Superfect (purchased from Qiagene) was added to the centrifuge tube and allowed to stand for 8 min at 25° C.

(5) The DNA-Superfect mixture in the centrifuge tube was added dropwise to the cultured cells and vortexed.

(6) The system was incubated for 5 h at 37° C. in a CO₂ incubator.

(7) The solution in the culture medium was aspirated and removed, the culture medium was washed with AIM-V (BRL), and new AIM-V was added to continue incubation.

(8) The cells were placed back in the CO₂ incubator and cultured overnight. The transduction efficiency was observed on the next day.

### Example 5

This example conducted purification and concentration of lentiviruses.

### 1. Viral Vector Purification

Cell debris was removed through centrifugation (at 1000×g) to obtain a virus supernatant, the virus supernatant was filtered by a low protein binding filter, and the viruses were divided into small portions and stored at −80° C.

2. Lentivirus Vector Concentration with a Centrifugal Filter

(1) In a biosafety cabinet, a concentration tube was disinfected and washed under sterile conditions twice.

(2) The virus vector supernatant was added to each centrifugal filter tube and centrifuged until the virus volume was reduced by a factor of 30.

(3) The filter tube was oscillated and centrifuged, the concentrated viruses were collected into a collection cup, and the virus vectors in all tubes were pooled into one centrifuge tube.

### Example 6

This example provided two types of chimeric antigen receptor T cells. The chimeric antigen receptor T cells expressed the chimeric antigen receptor targeting PSMA (12313) in Example 2 and the control chimeric antigen receptor targeting LMP2 in Example 4. The preparation method is as follows:

The activated T cells were suspended in a culture solution, and polybrene (purchased from Sigma) of 10 μg/mL was added. The culture solution was AIM-V containing cell culture factors IL-2, IL-7, and IL-15 (purchased from Peprotech). The concentrated lentiviruses in Example 5 were added respectively. The cells were centrifuged for 100 min at 25° C. at 100 g and cultured for 24 h at 37° C. A culture solution was added. After four days of culture, the cells were harvested and counted. After two days of culture, the cells were safely detected and transferred to a patient.

### Example 7

This example conducted the in vitro killing assay of CAR-T cells (12313) associated with target cells and CAR-T cells (LMP2) not associated with target cells.

(1) Green fluorescent proteins were transferred into two PSMA-positive tumor cell lines, that is, a prostate tumor cell line LNCap2 and a multiple myeloma cell line Molp2, via lentiviral vectors for stable expression.

(2) LMP2 CAR-T cells were used as a negative control, and 12313 PSMA CAR-T cells in Example 6 were used as an experimental group. The above two types of CAR-T cells were co-cultured with the two tumors in step (1) for 1 to 8 days at 37° C. in a 5% CO₂ incubator. During the culture process, the killing of the tumor cells was observed via a fluorescence microscope and recorded daily. The results are shown in FIG. 2, and as can be seen, the killing effect of the PSMA CAR-T (12313) group was significantly superior to the killing effect of the control group (LMP2).

### Example 8

This example treated brain glioma using PSMA CAR-T cells (12313 CAR-T).

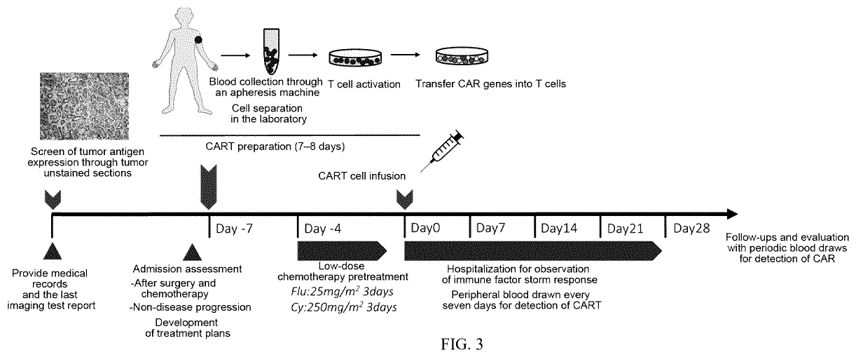

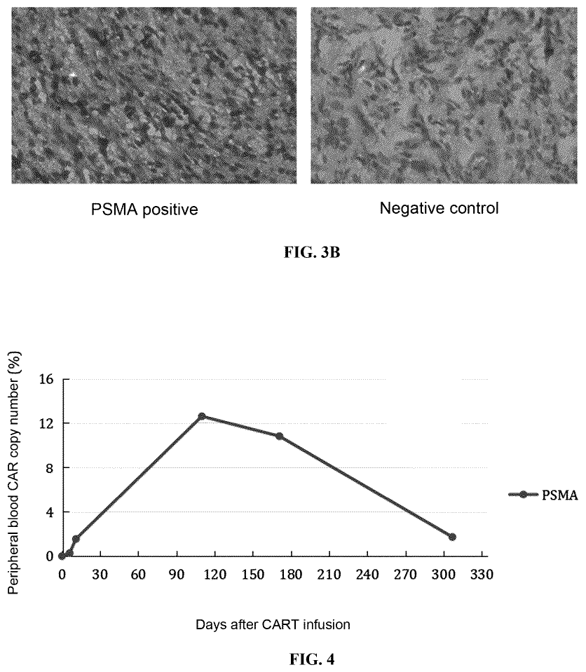

(1) One patient with refractory brain glioma was recruited as the subject, and the overall treatment flow is shown in FIG. 3A.

(2) The unstained tumor sections of the patient confirmed the positive expression of PSMA via immunohistochemical staining, as shown in FIG. 3B.

(3) The concentrate of white blood cells of the patient was collected. Peripheral mononuclear lymphocytes in the concentrate of white blood cells were separated through density gradient centrifugation with Ficoll, and T cells were screened out by CD3 magnetic beads and activated by adding an anti-CD28 antibody (purchased from BD Biosciences). PSMA CAR-T cells were prepared at 2×10⁶ CAR-T cells per kilogram of the body weight.

(4) Before infusion, the patient was pretreated with a small dosage of chemotherapy. The pretreatment regimen was cyclophosphamide (250 mg/m²) for three days and fludarabine (25 mg/m²) for three days. CAR-T cell infusion was conducted 24 h after the pretreatment, and the pretreatment cost a total of four days.

(5) CAR-T cells were infused intravenously.

(6) After infusion, the patient was monitored by the clinician and evaluated for cytotoxic response. The clinical cytotoxic response was cytokine release syndrome (CRS). The results show that no CRS response was observed in the patient.

(7) The tumor lesions of the patient were evaluated by MRI before and after infusion, and the patient was assessed to be in stable condition.

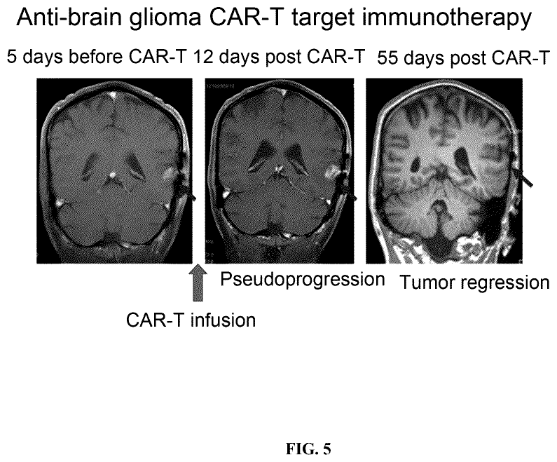

(8) A small amount of peripheral blood was drawn from the patient at regular intervals after infusion, the cell chromosome DNA (gDNA) was extracted after mononuclear lymphocytes were separated from the peripheral blood, and then CAR copy numbers in the peripheral blood were quantified using specific primers by qPCR (for specific operations, see Chang, L.-J. and Zaiss, A.-K. (2001) Methods for the preparation and use of lentivirus vectors. Methods in Molecular Medicine, Gene Therapy Protocols, 2nd Ed., pp 303-318, Ed. Jeffrey Morgan, Humana Press, Inc.). FIG. 4 shows the curve graph of changes in PSMA CAR copy numbers in the patient with glioblastoma obtained after CAR copy numbers in the blood drawn from the patient infused with CAR-T cells were detected. FIG. 5 shows the MRI image of the brain of the patient with brain glioma before and after the infusion of CAR-T cells when the patient was clinically infused with PSMA chimeric antigen receptor T cells for the treatment of brain glioma. The results show that the tumor image was significant five days (−5) before the infusion of the CAR-T cells, the tumor in the pseudoprogression grew larger due to the infiltration of the CAR-T cells 12 days after the infusion of the CAR-T cells, and the tumor shrank 55 days after the infusion of the CAR-T cells.

In summary, the PSMA chimeric antigen receptor of the present application has better response effects and long-term effectiveness. The PSMA chimeric antigen receptor, when being applied in patients with glioblastoma expressing the tumor-specific target PSMA, has fewer clinical side effects and higher safety, and can effectively eliminate minimal residues that are insensitive to chemotherapy. In addition, the PSMA CAR-T cells are also used in combination with other targeted CAR-T cells in patients with brain glioma, and the presence of the PSMA CAR-T cells is monitored in patients in vivo for a long period of time, which is conducive to maintaining long-term remission.

The applicant has stated that although the detailed method of the present application is described through the embodiments described above, the present application is not limited to the detailed method described above, which means that the implementation of the present application does not necessarily depend on the detailed method described above. It is to be apparent to those skilled in the art that any improvements made to the present application, equivalent replacements of raw materials of the product of the present application, additions of adjuvant ingredients, selections of specific manners, etc., all fall within the protection scope and the disclosure scope of the present application.

1. An anti-prostate-specific membrane antigen (anti-PSMA) single-chain antibody, comprising:
a heavy chain, wherein an amino acid sequence of the heavy chain comprises a sequence having 80% or more identity to SEQ ID NO: 1; and
a light chain, wherein an amino acid sequence of the light chain comprises a sequence having 80% or more identity to SEQ ID NO: 2.

2. A nucleic acid molecule, comprising a nucleic acid sequence encoding the anti-PSMA single-chain antibody of claim 1.

3. A PSMA chimeric antigen receptor, comprising an antigen binding domain, a transmembrane domain, a costimulatory signaling domain, and a CD3ζ signaling domain;
wherein the antigen binding domain comprises the anti-PSMA single-chain antibody of claim 1.

4. The PSMA chimeric antigen receptor of claim 3, wherein the transmembrane domain comprises a CD28 transmembrane domain and/or a CD8α transmembrane domain;
preferably, the costimulatory signaling domain comprises a CD28 signaling domain and a CD27 signaling domain or comprises a CD28 signaling domain and an IL-15Ra signaling domain;
preferably, the PSMA chimeric antigen receptor comprises a CD28 transmembrane domain, a CD28 signaling domain, and a CD27 signaling domain;
preferably, the PSMA chimeric antigen receptor comprises a CD28 transmembrane domain, a CD28 signaling domain, and an IL-15Ra signaling domain;
preferably, the PSMA chimeric antigen receptor further comprises a suicide-inducing fusion domain;
preferably, the suicide-inducing fusion domain comprises a caspase 9 domain fused with an FK506 binding protein (FKBP);
preferably, the PSMA chimeric antigen receptor further comprises a signal peptide and/or a 2A sequence;
preferably, the signal peptide comprises a Secretory signal peptide; and
preferably, the PSMA chimeric antigen receptor comprises a Secretory signal peptide, an antigen binding domain, a transmembrane domain, a costimulatory signaling domain, a CD3ζ signaling domain, a 2A sequence, and a suicide-inducing fusion domain.

5. A viral vector, comprising a nucleic acid molecule encoding the PSMA chimeric antigen receptor of claim 3;
wherein preferably, the viral vector comprises a lentiviral vector or a retroviral vector and preferably, is a lentiviral vector.

6. A recombinant virus, obtained by co-transduction of the viral vector of claim 5 and packaging helper plasmids into a mammalian cell;
wherein preferably, the packaging helper plasmids comprise pNHP and pHEF-VSVG; and
preferably, the mammalian cell comprises any one of a 293 cell, a 293 T cell or a TE671 cell.

7. A chimeric antigen receptor cell, expressing the PSMA chimeric antigen receptor of claim 3;
wherein preferably, the chimeric antigen receptor cell is prepared by transduction of a nucleic acid molecule encoding the PSMA chimeric antigen receptor of claim 3 into an immune cell;
preferably, a manner of the transduction comprises any one of transduction by a viral vector, transduction by a eukaryotic expression plasmid or transduction by mRNA and preferably, is transduction by a viral vector; and
preferably, the immune cell comprises a T cell.

8. A composition, comprising any one or a combination of at least two of the anti-PSMA single-chain antibody of claim 1.

9. (canceled)

10. The method of claim 11, wherein the tumor comprises a tumor expressing a PSMA-specific antigen;
preferably, the tumor comprises a hematological tumor expressing a PSMA-specific antigen and a solid tumor expressing a PSMA-specific antigen; and
preferably, the tumor comprises a prostate cancer, a lymphoma, a multiple myeloma, a renal cancer, a bladder cancer, a colon cancer, a neuroblastoma or a brain tumor.

11. A method for treating a tumor, comprising administering an effective amount of the anti-PSMA single-chain antibody of claim 1 to a patient in need thereof.


SEQUENCE LISTING (15 sequences)

SEQ ID NO: 1 - protein, 115 aa, synthetic construct
EVQLVQSGAEVKKPGASVKISCKISGYTFTEYTIHWVKQASGKGLEWIGNINPNNGGTTYNQKFEDRATLTVDKSTSTAYMELSSLRSEDTAVYYCAAGWNFDYWGQGTTVTVSS

SEQ ID NO: 2 - protein, 107 aa, synthetic construct
DIVMTQSPSSLSASVGDRVTIICKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 3 - DNA, 720 bp, synthetic construct
  source 1..720: mol_type = other DNA
gaggtccaactggtgcagtctggtgctgaagtgaagaagcctggtgccagcgtgaagattagctgcaagattagcggctacaccttcaccgagtataccattcactgggtcaaacaagcctctggaaaagggctggaatggataggcaatatcaaccccaacaatggcggaacaacctacaaccaaaagtttgaggatcgtgccactctgacggttgacaagtccacgagcacagcctacatggagctgtcaagtctgaggtccgaggatactgcggtctactattgtgctgctgggtggaacttcgactattggggacagggtacgacagttactgtgtcctcaggctcaacatccggctctggaaagcctgggtccagtgaagggagcacaaaaggggacatcgtgatgacccagtctccaagcagccttagcgctagtgtaggggatcgagtgaccatcatctgcaaagcatctcaggacgtaggcactgcagtggattggtatcagcagaaaccaggaaaagcgccgaaactgttgatctactgggcaagtacacgccacactggagtcccagatcggtttaccgggtccggctcaggcactgacttcactctgaccatttcctctcttcagcccgaagattttgccgactacttctgtcagcagtacaatagctatcccctgacatttggcggtggaacaaagctcgagataaag

SEQ ID NO: 4 - protein, 167 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSASGGGGSGGGGSQRRKYRSNKGESPVEPAEPCHYSCPREEEGSTIPIQEDYRKPEPACSP

SEQ ID NO: 5 - protein, 158 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSASGGGGSGGGGSKSRQTPPLASVEMEAMEALPVTWGTSSRDEDLENCSHHL

SEQ ID NO: 6 - protein, 401 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGGGGSGAMVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 7 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 24 aa, synthetic construct
TSGSGATNFSLLKQAGDVEENPGP

SEQ ID NO: 9 - protein, 240 aa, synthetic construct
EVQLVQSGAEVKKPGASVKISCKISGYTFTEYTIHWVKQASGKGLEWIGNINPNNGGTTYNQKFEDRATLTVDKSTSTAYMELSSLRSEDTAVYYCAAGWNFDYWGQGTTVTVSSGSTSGSGKPGSSEGSTKGDIVMTQSPSSLSASVGDRVTIICKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 10 - protein, 21 aa, synthetic construct
MALPVTALLLPLALLLHAARP

SEQ ID NO: 11 - protein, 22 aa, synthetic construct
MLLLVTSLLLCELPHPAFLLIP

SEQ ID NO: 12 - protein, 18 aa, synthetic construct
GSTSGSGKPGSSEGSTKG

SEQ ID NO: 13 - protein, 5 aa, synthetic construct
GGGGS

SEQ ID NO: 14 - protein, 10 aa, synthetic construct
GGGGSGGGGS

SEQ ID NO: 15 - protein, 15 aa, synthetic construct
GGGGSGGGGSGGGGS